(19) United States
(12) Patent Application Publication
(10) Pub. No.: US 2011/0106240 A1
(43) Pub. Date: May 5, 2011

(54) LARGE VESSEL STENTS AND OCCLUDERS

(76) Inventor: Timothy A.M. Chuter, Atherton, CA (US)

(21) Appl. No.: 12/945,679

(22) Filed: Nov. 12, 2010

Related U.S. Application Data
(63) Continuation of application No. 10/018,164, filed on May 20, 2002, now abandoned.
(60) Provisional application No. 60/186,806, filed on Mar. 3, 2000.

(51) Int. Cl. A61F 2/82

(52) U.S. Cl. 623/1.22; 623/1.31

(57) ABSTRACT

An endovascular stent for vascular vessels which can be used to occlude the vessel or which can be used to bridge damaged areas in the vessel. The endovascular stent comprising a stent that can be permanently expanded from a first diameter to a larger second diameter. The stent can be a helically wound wire stent, each wire comprising at least two strands, the two strands being twisted. The twisted strands securing fibers to form a fabric pile extending outwardly from stent, and optionally extending inwardly into the stent. In a second embodiment, the stent is enclosed with a tubular-like expandable graft, the graft having an exterior fabric pile made up of individual fibers. In both embodiments, the fibers or the pile are optionally coated with a hydrophilic polymeric gel which expands upon being wetted.

### FIELD OF THE INVENTION

This invention relates to intervascular stents for maintaining vascular patency in humans and animals, and to intervascular stents for occluding vascular members in humans and animals, and to hydroscopic plugs or occluders for vascular members.

### BACKGROUND OF THE INVENTION

Over the last fifteen years, great advances have been made in vascular surgery and treatment, including angioplasty balloon dilation of elastic vascular stenosis, application of a catheter mounted angioplasty balloon and intralumenal endovascular grafting employing intralumenal vascular grafts and stents.

The patents on endoprosthetsis devices, most commonly called stents, is extensive and includes the following U.S. Pat. Nos. 4,503,569; 4,553,545; 4,580,568; 4,655,771; 4,733,665; 4,739,762; 4,830,003; 4,886,062; 4,913,141; 4,990,155; 5,015,253; 5,019,085; 5,019,090; 5,037,427; 5,104,404; 5,133,732; 5,135,536; 5,222,971; 5,226,913; and 5,370,683. The disclosures of these identified patents is hereby incorporated by reference. A number of prior art stents can be employed in the present invention including the stents disclosed in the Dotter U.S. Pat. No. 4,503,569; the Gianturco U.S. Pat. No. 4,580,568; the Wallsten U.S. Pat. No. 4,655,771; the Palmaz U.S. Re-Examination Certificate Pat. No. B1 4,733,665; the Palmaz U.S. Pat. No. 4,739,762; the Hillstead U.S. Pat. No. 4,913,141; the Wilkoff U.S. Pat. No. 4,990,155; Wiktor U.S. Pat. No. 4,886,062; the Fontaine U.S. Pat. No. 5,370,683; the MacGregor U.S. Pat. No. 5,015,253; Hillstead U.S. Pat. No. 5,019,085; Pinchuk U.S. Pat. No. 5,019,090; Haraka et al U.S. Pat. No. 5,037,427; Wolff U.S. Pat. No. 4,104,404; Wiktor U.S. Pat. No. 5,133,732; Hillstead U.S. Pat. No. 5,135,536; Willard et al. U.S. Pat. No. 5,222,971; Pinchuk U.S. Pat. No. 5,226,913; and Maass et al. U.S. Pat. No. 4,553,545.

The vascular system of humans and animals is a complex system made up of arteries, veins and capillaries. These vessels bend and curve through the body and have a generally circular shapes, but, cross sectional shapes of the vessels for a variety of reasons can be far from an idealized circular cross sectional area. In just a matter of a centimeter, a large vessel can change from a relatively circular cross sectional area to a markedly oval shape, then to a cusp cross sectional shape, and so on and so forth. It is not possible to custom make stents to fit a particular area of the vascular system and it is not possible to manufacture stents of all shapes and sizes and lengths to provide a stent to fit each vascular system demand. Although the vascular system is relatively flexible, the areas requiring a prosthesis to repair vessels narrowed or occluded by disease, such as stenosis, restrictions, aneurysms, lesions, plaque, and the like, are not flexible. In those areas that are diseased, a vessel is relatively inflexible and an expanding stent will not reshape the vessel into a circular cross section to obtain a good fit between the expanded circular cross section stent and the interior wall of the vascular vessel. When the stent does not fit well within the interlumenal passageway of the vessel, blood can flow between the outer surface of the stent and the interlumenal surface of the vessel causing an area of turbulence which gives rise to clotting. Frequently these clots are not anchored securely and break free, and circulate to vital organs such as the lungs or brain.

Although stents are normally employed to enhance the patentcy of a vascular vessel, there are occasions when the vascular surgeon wishes to occlude a vascular vessel, such as when a severely damaged vessel is surgically bypassed with a bypass vessel. After the bypass vessel has proven to surgically taken at the point of incisions, the diseased and damaged portion of the vessel is then occluded to prevent future problems with that portion. In this instance, it is extremely important to cut off and occlude the damaged portion of the vessel to prevent all blood flow into it and through it to prevent future complications. Because of the diseased nature of the damaged portion of the vessel, the vessel is frequently very inflexible and has a very irregular shape which is not well adapted for employing tubular stent in an attempt to block off the vessel.

Abdominal aortic aneurysm is a dilation of the distal aorta, which can lead to rupture and fatal intra-abdominal hemorrhage. Conventional treatment involves replacement of the dilated segment with a durable fabric conduit, or graft. This is an effective treatment, but it involves major painful, debilitating and expensive surgery. An alternative endovascular method of treatment has recently been developed in which a graft is introduced in a remote artery and positioned and secured in the damaged portion of the aortic artery by the expansion of a metallic lattice, or stent, thereby isolating and occluding the aneurysm from aortic circulation and preventing rupture.

One of the commoner forms of endovascular exclusion involves implantation of a stent-graft from the aorta to the iliac artery in the side of the insertion. This leaves the other iliac artery as a potential route for arterial blood flow into the aneurysm unless the repair is also accompanied by some means of inducing iliac artery occlusion. Unfortunately, the iliac arteries are often large and irregular in patients with dilation of the aorta and none of the current endovascular devices that already exist are for the occlusion of small to medium size arteries are suited for treatment of aneurysms in the iliac artery.

Stent-graft combinations and detachable balloons have also been used as arterial occluders. Unfortunately, these combinations and balloons have not fulfilled their role as arterial occluders very well.

Stent grafts are inserted with one or both ends of the graft sewed shut. If the stent has a high expansion ratio, that is, if it will expand radially outward from a first smaller diameter to a second larger diameter, then the stent graft is constructed to a thin-wall fabric, and it is possible to deliver a stent-graft large enough to occlude most iliac arteries. However, the constant diameter cylindrical profile of a stent-graft usually prevents the stent-graft from closing off the artery because of the surface irregularities commonly seen in the recipient artery which is already damaged. In most instances, gaps remained between the stent-graft and portions of the arterial inner wall. These gaps allow leakage of blood between the stent-graft and the artery. Thus the stent-graft fails to accomplish the purpose of damming off or walling off the aneurysm and the gap between the exterior surface of the stent-graft and the interior arterial wall frequently leads to complications resulting either from clot formation in the gap which escape from the gap and enter the lungs or delamination of the interior arterial wall surface.

Detachable balloons used for arterial occlusion suffer from several limitations. Several balloons are normally required to fill large arteries like the large iliac arteries commonly encountered in association with an aortic aneurysm. The balloons normally deflate with time leading to recurrent aneurysm perfusion, thus defeating the purpose of the balloon insertion.

### SUMMARY OF THE INVENTION

The present invention is directed to the use of an expanding stent, either self expanding or expandable, and a fiber pile on the outside of the stent to yield a stent which can be employed as an endovascular prosthesis for the repair of a damaged vascular vessel and/or for bridging damaged and/or diseased areas of a vascular vessel. The fiber pile is similar to carpet pile. In addition, the invention is directed to the use of expandable stents and a fabric pile to yield a endovascular occluding stent for sealing off a vascular vessel to induce the thrombosis of large arteries to seal off portions of the artery, especially damaged portions. Moreover, the present invention is directed to the use of expandable stents with a fabric pile exterior or sheath where the fabric pile is coated with an expandable hydrophilic gel material. Furthermore, the invention is directed to a vascular plug employing hydrophilic material in a bag.

The endovascular prosthesis of the present invention comprises an expandable stent supporting an external elastic fabric pouch like graft, the stent adapted to be permanently expanded from a first diameter adapted to permit the vascular surgeon to position the stent into the desired area to a larger second diameter to cause contact of the outer circumferential wall of the stent with the lumenal wall of the vascular vessel and the graft, the graft adapted to radially expand with the stent, the graft having a fiber pile on its external surface about the tubular side wall of the stent, the graft made of fiber adapted to form a foundation for tissue growth between the lumenal wall and the endovascular prosthesis to incorporate the endovascular prosthesis with the vascular vessel. Virtually any expanding stent can be used in this embodiment.

The endovascular prosthesis of the present invention can also be employed to occlude the lumen of a vascular vessel. The endovascular prosthesis comprises a generally tubular shaped expandable stent adapted to be permanently expanded from a first diameter which is suitable for insertion of the stent into the lumen of a vascular vessel to a larger second diameter to obtain contact with the lumenal wall of the vascular vessel and a cylindrical flexible graft having an open end and closed opposing end. The cylindrical flexible graft is adapted to radially expand with the stent. The closed end of the cylindrical graft extending over one end of the tubular stent and sealing off the end of the stent. The graft having a fabric pile adapted to form a foundation for tissue growth between the fabric pile of the graft and lumenal wall of the vascular vessel. The graft adapted to induce thrombosis between the lumen of the vascular vessel and the prosthesis to occlude said vessel. Virtually any expanding stent can be used in this embodiment. The flexible graft can also have both ends closed.

The endovascular prosthesis can also comprise a generally conical-shaped expandable stent adapted to be expanded from a first diameter which is suitable for insertion of the stent into a lumen of a vascular vessel to a larger second diameter to obtain contact with the lumenal wall of the vascular vessel and a generally conical-shaped flexible graft having an open end and a closed opposing end. The generally conical-shaped flexible graft is adapted to radially expand with the stent. The generally radially shaped expandable stent has one end with a smaller diameter and the opposing ends with a larger diameter. In the preferred embodiment, the stent prior to radial expansion has close to a tubular shape and expansion is progressively greater at one end, the larger diameter end, of the stent. Similarly, the generally conical-shaped flexible graft has a larger diameter end and an opposing smaller diameter end. Normally, the smaller diameter opposing end is the one that is sealed off. However, both ends of the cylindrical graft can be closed off. The graft has a fabric pile adapted to form a foundation of tissue growth between the fabric pile of the graft and the lumenal wall of the vascular vessel.

In another embodiment, the fabric pile of the above stent graft can be coated with a pharmaceutically acceptable hydrophilic polymeric gel. The stent graft with such a coating is utilized in the vascular system in at least a partially dehydrated state. The body fluids, primarily blood, will hydrate the hydrophilic polymer gel, expanding the gel to aid in further sealing any gaps between the lumenal wall of the vascular vessel and the stent graft. The fabric pile can be coated with the hydrophilic gel so that each strand of the fabric pile is coated, or the fabric pile can be encapsulated in a thick layer of the hydrophilic gel which completely surrounds the fabric pile, or the strands of the fabric pile can have one or more beads of hydrophilic gel attached to the strands.

In another embodiment, the endovascular prosthesis can comprise a generally tubular shape expandable stent adapted to be permanently expanded from a first diameter which is suitable for insertion of the stent into the lumen of a vascular vessel to a larger second diameter to obtain contact with a lumenal wall of the vascular vessel and a cylindrical flexible graft covering the tubular side wall of the stent and having open ends at both ends. The cylindrical flexible graft is adapted to radially expand with the stent. The cylindrical graft only covers the outer tubular wall of the stent leaving the ends of the stent open to permit the flow of blood. This endovascular prosthesis can be used to bridge an aneurysm or other damaged area of a vessel to permit blood to flow and bypass the damaged area.

The endovascular stent for the repair of vascular vessel can also comprise a generally tubular stent member which can be made of at least one helically wound wire, each wire comprised of at least two twisted strands, the twisted strands securing fibers, the fibers extending radially outward from the stent to form a fiber pile, the fiber pile adapted to form a foundation for tissue growth between the fiber pile and the lumenal wall of a vascular vessel. The twisted strands can also secure fibers extending radially inwardly to form an inner fiber pile. The fibers of this endovascular stent can be coated with a pharmaceutically acceptable hydrophilic gel as described above.

The endovascular stent for occluding the lumen of a vascular vessel can comprise a generally conical member having a lesser diameter at one end and a larger diameter at the other end made of at least one helically wound wire, the wire comprising of at least two twisted strands, the twisted strands securing fibers to form a fiber pile extending outwardly from the stent and optionally extending inwardly into the stent to substantially occlude the inner bore of the stent. Prior to permanently radially expanding the stent, the stent preferably has a more tubular shape than conical shape and expansion occurs more progressively at one end, the larger diameter end, of the stent. The fiber pile forming a foundation for tissue growth between the fiber pile and the surrounding lumenal wall of the vascular vessel and for tissue growth in the bore of the stent to cause occlusion of the vessel.

The endovascular stent can be comprised of two or more wire helicals, with one group of wires wound in one direction, such as left hand direction, and the other group of wires wound in the opposite direction, that is the right hand direction, to form an interweaving structure stent. Optionally, the helically wound wires can be woven or braided so that a particular wire crosses over and crosses under other wires in a predetermined pattern.

Another embodiment of the present invention, the endovascular prosthesis is a stent for occluding a vascular lumen comprising a generally umbrella-shaped member having a plurality of radial wire ribs biased to extend radially outward, each wire ribs comprising of at least two twisted strands. The twisted strands supporting and securing fibers which extend outwardly from the wire ribs to form a fiber pile, the fiber pile adapted to form a foundation for tissue growth between the fiber pile and the entire circumferential lumenal wall surface. Optionally, fibers can also extend inwardly to form a fiber pile in the interior of the stent. The fibers of this endovascular stent can be coated with a pharmaceutically acceptable hydrophilic gel as described above.

The stents that can be employed in the present invention include self expanding stents which are inserted into the lumen of the vascular vessel in a compressed state and when released, expand on their own. Alternatively, stents can be employed which can be expanded either employing balloons or employing stents which are rotated about their longitudinal axis or contracted along their longitudinal axis to increase the diameter of the stent.

The fibers of the fiber pile are biocompatible fibers which are known to the art. Suitable fibers include nylon fibers, polyester fibers, Mylar brand fibers and the like. The pharmaceutically acceptable hydrophilic gels are polymeric materials, either natural or synthetic, which are compatible with mammal body tissues and fluids. The pharmaceutically acceptable hydrophilic gels can be fully dehydrated for insertion into the vascular vessel, or they can be partially hydrated for insertion into the vascular vessel. When the graft stent having a coating of hydrophilic gel is positioned within the vascular vessel, the body fluids, primarily blood, will hydrate the hydrophilic gel completely to fully expand the gel. The hydrophilic gel is not soluble in the body fluids or blood. The hydrophilic gel is nontoxic. The hydrophilic gel adheres to the fibers of the fiber pile by mechanical and/or chemical adherence.

In the preferred embodiment of the present invention, the fiber pile is coated with a hydrophilic polymeric gel. The fiber pile is coated with a polymeric hydrophilic gel which is allowed to partially dry. The coated fibers are then “combed” downwardly to reduce the overall outer diameter of the stent to the greatest extent possible for ease of insertion through a catheter into the lumen of the vascular vessel. The partially dried gel is then preferably fully dried to reduce the volume of the gel to the greatest extent. The stent with the fiber pile and dried gel coating are sterilized in the conventional manner. When the surgeon prepares the stent for insertion into the lumen of the vascular vessel, the dried gel can be wetted with sterile saline or water which partially hydrates the gel and lowers its coefficient of friction for insertion into the vascular lumen. In its final place of disposition in the vascular system, the gel absorbs water from blood, blood serum, blood plasma, and the like. Prior to expanding the stent from the first diameter to the second diameter, the stent can be maintained in position for a few minutes to allow the gel to more fully hydrate in the fluid environment. As the gel to becomes more fully wetted, it expands in volume. Preferably, the gel promotes thrombosis in order to aid in the sealing and securing the surface of the stent graft to the internal lumenal wall. The thrombosis also encourages tissue growth so that eventually the stent and the graft become incorporated into the wall of the vascular vessel. A biologically acceptable nontoxic hydrophilic gel is employed in the present invention, such as hydrophilic acrylates, polyvinyl pyrolidones, carboxylic acrylic polymers and co-polymers, polyurethanes and natural gels known to the art. Suitable gels are identified in U.S. Pat. Nos. 5,331,027; 5,443,907; and 5,490,839. The disclosures of these patents are incorporated herein by reference.

Fiber of the fabric pile and the gel are selected so that the gel remains adhered to the fibers and does not migrate away from the fibers into the bloodstream. Most of the biological fibers are made of polymeric materials which are not highly polarized. Accordingly, such fibers frequently have to be coated with a primer which adheres to the fiber and yet has a polar constituent which attracts the polar constituents of the gel. Alternatively, the fibers can be treated with electric discharge or plasma discharge before being coated with the gel to present a polarized surface environment to attract and secure the gel to the fiber.

The stents of the present invention that employ a graft can extend the full length of the graft. The graft pouch, sock or sleeve can extend beyond one end or both ends of the stent. The graft pouch or sock, or can be shorter or longer than the stent.

In another embodiment of the present invention, vascular vessel is occluded with a bag filled with a solid. In one embodiment, the bag is semi-permeable and is inserted into the vascular vessel in the empty state through a catheter. When bag is positioned, a slurry of particulate solid and saline or water is pumped through a delivery tube attached to the bag. The slurry comprises pharmaceutically acceptable materials. Some of the particles are preferably at least a partially dehydrated hydrophilic gel or a water-activated cement. An expandable impervious bag can also be employed. The impervious bag can be filled with polymeric gel aqueous slurry which expands and gels upon sitting, occluding the vessel. The empty bag attached to the end of a delivery tube is inserted into and positioned in the vascular vessel through a catheter. The slurry is pumped into the deflated bag and allowed to set up to form an expanded insoluble mass. The polymeric gel material is pharmaceutically acceptable and compatible with the body's tissues and fluids. After the polymeric material is set up into a solid mass, the delivery tube is disconnected from the bag and removed from the vascular system. The bags are filled and expanded to the point where they occlude the vascular vessel and form a permanent wall which seals off the vascular vessel.

In an alternative embodiment of the present invention, the occluder comprises a dual bag construction with an outer elastic impervious bag and an inner semi-pervious elastic bag. The dual bag is attached to a delivery tube for filling the inner semi-permeable bag. After the bag has been positioned in the arterial vessel, water or saline solution is delivered through the delivery tube into the inner bag to expand the at least partially dehydrated hydrophilic polymeric gel granules in the bag by hydration. As the gel granules expand from hydration, the inner bag expands against the inner wall of the outer bag and expands the outer bag to come in contact with the inner wall of the arterial vessel to occlude the vessel. After the inner bag is fully hydrated and expanded to its maximum dimensions, the delivery tube is separated from the dual bag and removed through the catheter from the arterial system.

### BRIEF DESCRIPTION OF THE DRAWINGS

FIG. 1 is a perspective view of a graft of the present invention;

FIG. 2 is a cross-sectional view of FIG. 1;

FIG. 3 is an enlarged sectional view of FIG. 2;

FIG. 4 is a cross-sectional view of an occluder of the present invention;

FIG. 5 is a partial perspective view of another occluder of the present invention;

FIG. 6 is a partial enlarged view of circled line 6 of FIG. 5;

FIG. 7 is a enlarged cross-sectional view taken along lines 7-7 of FIG. 6;

FIG. 8 is a perspective view of another occluder of the present invention;

FIG. 9 is an end view of the occluder of FIG. 8;

FIG. 10 is an enlarged cross-sectional view taken along lines 10-10 of FIG. 9;

FIG. 11 is a cross-sectional view of another embodiment of the occluder of FIG. 4;

FIG. 12 is a cross-sectional view of another embodiment of the occluder similar to the occluder of FIG. 8;

FIG. 13 is a cross-sectional view of another embodiment of graft of the present invention similar to the graft of FIG. 1;

FIG. 14 is an end view of another embodiment of the graft of FIG. 1;

FIG. 15 is a fragmentary side view of another embodiment of the occluder of the present invention;

FIG. 16 is an end view of the stent frame of the occluder of FIG. 15;

FIG. 17 is a cross-sectional view showing the insertion and positioning of the occluder of FIG. 15 into an arterial system;

FIG. 18 is a cross-sectional view of the occluder of FIG. 15 positioned in an arterial system;

FIG. 19 is a cross-sectional view showing the insertion and positioning of another occluder of the present invention into an arterial system;

FIG. 20 is cross-sectional view showing the placement of the occluder of FIG. 19 into the arterial system;

FIG. 21 is a cross-sectional view showing the final placement of the occluder of FIG. 19 into the arterial system;

FIG. 22 is another embodiment of the occluder of the present invention;

FIG. 23 is a cross-sectional view showing the occlusion of one end of an aneurysm in an arterial system with the occluder of the present invention;

FIG. 24 is a cross-sectional view showing the placement of the occluder of FIG. 23 in the arterial system;

FIG. 25 is a cross-sectional view showing the sealing off of an aneurysm with the occluders of FIG. 23; and

FIG. 26 is a cross-sectional view showing the bridging of an aneurysm with the stent graft of the present invention.

### DETAILED DESCRIPTION OF THE INVENTION

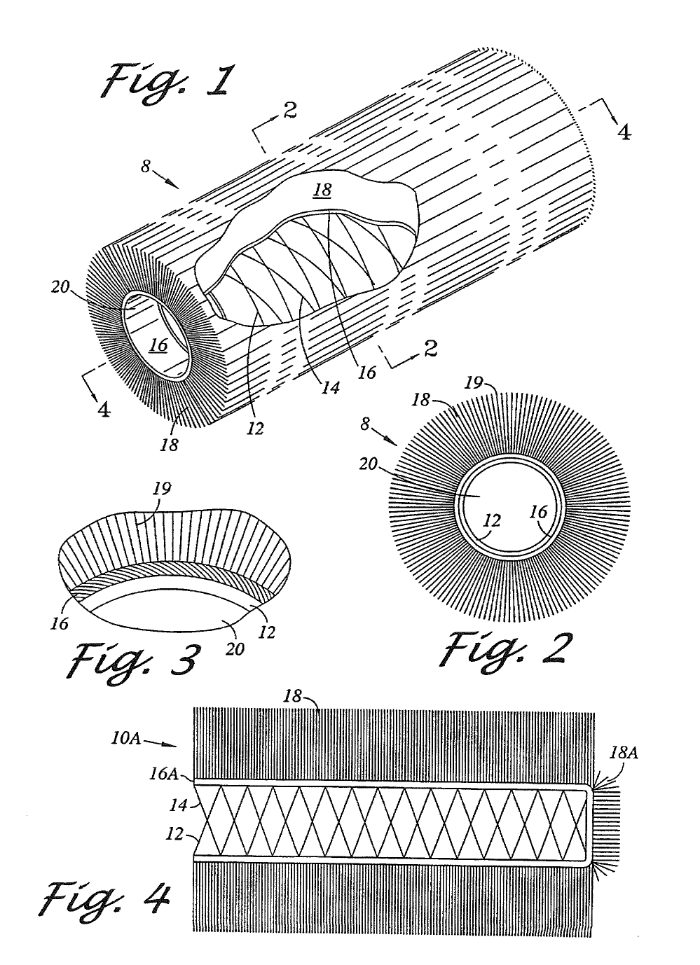

Referring to FIGS. 1-3, the graft 8 comprises a stent 12 comprising wires 14 helically wound into a stent frame supporting a fabric pile backing 16. Extending circumferentially outwardly from the backing 16 is a fabric pile 18 made up of individual fibers 19. The graft has a longitudinal lumen or bore 20 extending its length to permit blood flow.

Referring to FIG. 4, the occluder 10A comprises a stent 12 having wires 14 forming a stent frame which supports a sock fabric pile backing 16A. A fabric pile 18 made up of individual threads extends circumferentially from the longitudinal portion of the fabric pile backing and fabric pile 18A made up of fiber threads extending from the end of the fabric pile backing sock.

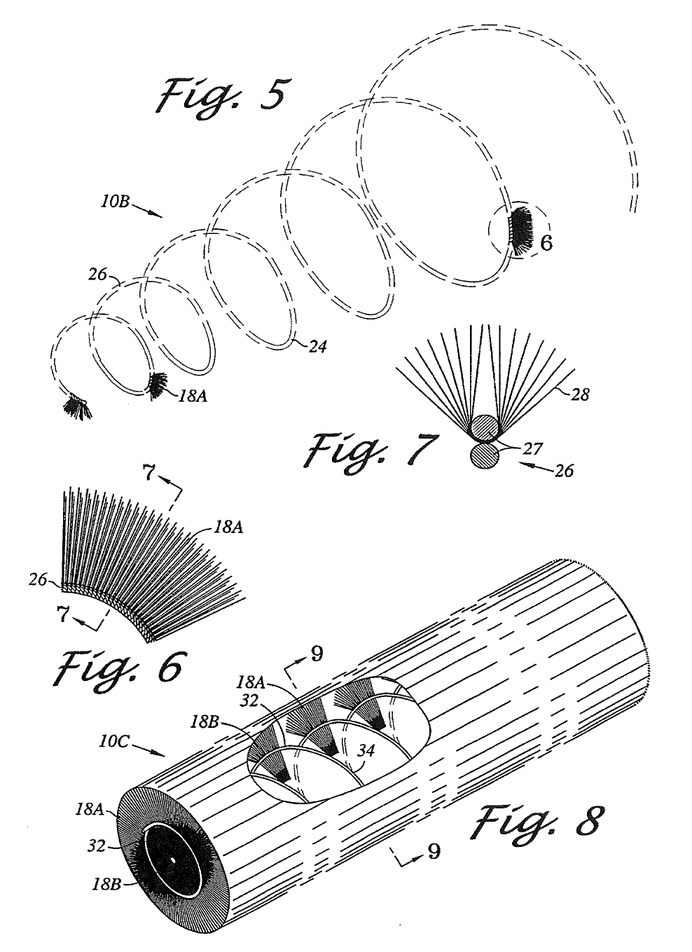

Referring to FIGS. 5-7, the conical shaped occluder 10B comprises a conical shaped stent 24 comprising at least one helically wound double wire strand 26. The wire strand 26 comprises of at least two wires 27 which are twisted (not shown). Fabric thread 28 which makes up the fabric pile 18A is inner-disposed between the wire strands 27 so that the fabric pile extends outwardly from the stent to form a conical shaped fabric pile device.

Referring to FIGS. 8-10, the occluder 100 comprises a stent 32 made up of a helical wire frame and comprising a wire strand 34 similar in cross section to the wire strand 26 shown in FIG. 7. However, the wire strand has fabric threads 28 extending both outwardly and inwardly circumferentially of the stent (see FIGS. 9 and 10) to form fabric piles extending outwardly from the occluder and inwardly of the stent to form a fabric pile “plug.”

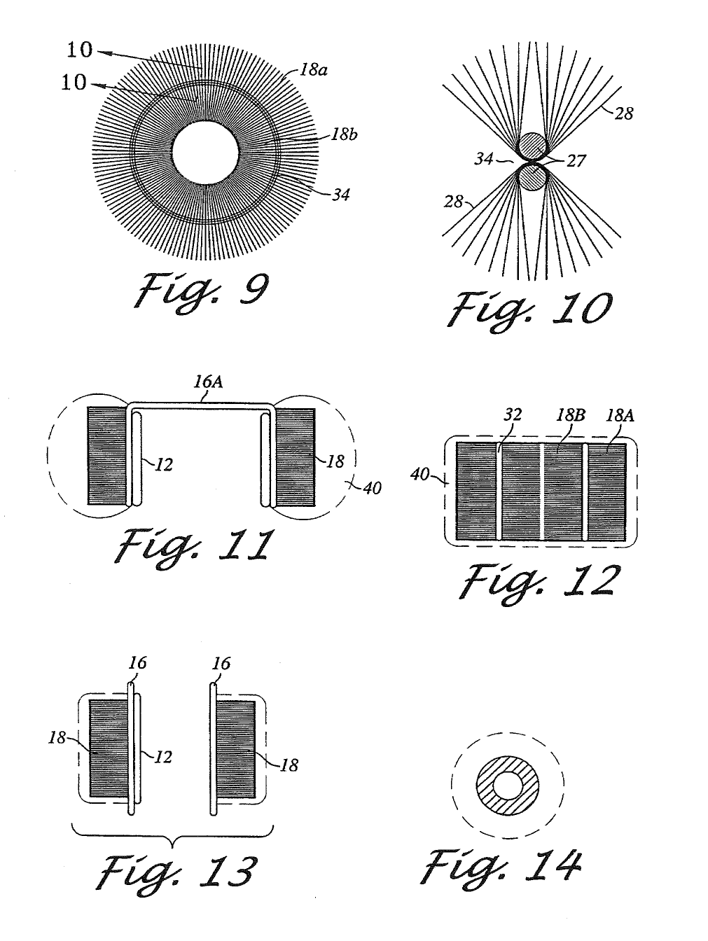

Referring to FIGS. 11-13, these figures schematically illustrate in cross section the occluder of FIG. 4 and FIGS. 8-10 and the graft of FIG. 1-3. The fabric pile is coated with hydrated hydrophilic gel 40 shown in phantom. After the gel is applied and saturated into the fabric pile, the gel is partially dehydrated by conventional means, such as elevated temperature preferably under a vacuum to dehydrate the gel, which makes the gel shrink and thicken. The gel can be further dehydrated to a dry state. Suitable hydrophilic gel is described in the summary of the invention. Prior to inserting the occluder or graft into the vascular system, the occluder or graft is wetted in order to partial hydrate the gel to make the outer surface of the occluder or graft more slippery. Once the occluder or graft is positioned in the vascular system through a catheter, the occluder or graft is positioned into the appropriate area of the arterial system employing a catheter, water from the blood stream, blood serum and/or blood plasma fully hydrates the gel. The gel fills any gaps between the inner lumina wall of the arterial system and the outer surface of the fabric pile to form a seal between the luminal wall and the occluder or graft.

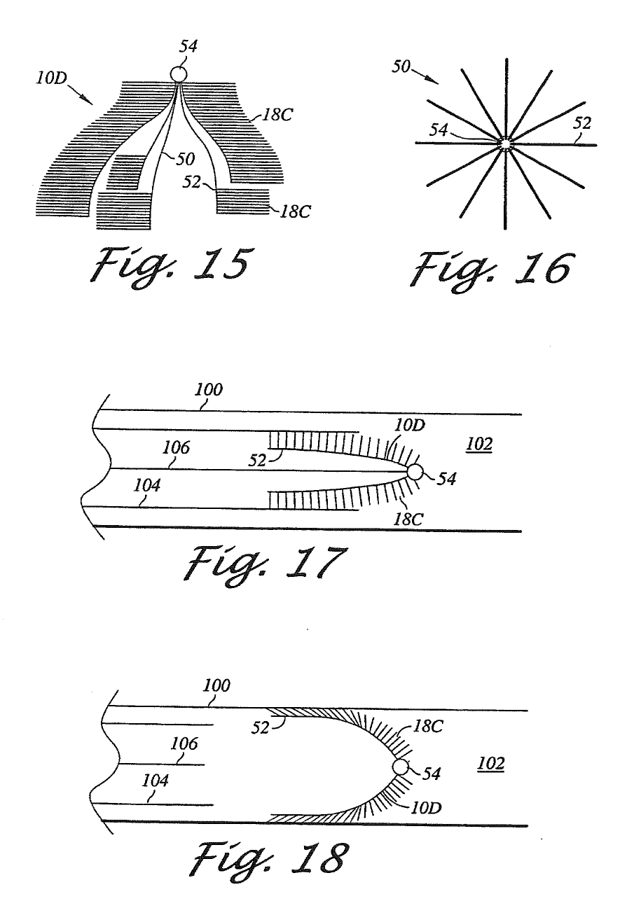

FIGS. 15-18 show another embodiment of the occluder of the present invention. This is an umbrella stent occluder 10D. The occluder 10D has a generally umbrella-shape; the supporting stent 50 has a plurality of ribs 52 radially extending from a central body 54. The ribs are biased to extend outwardly like extending umbrella ribs in its full open position. Each rib is made up of at least two twisted wires similar in cross section shown in FIG. 7. The twisted wires support therebetween fabric threads 28 which form the fabric pile 18C of the occluder. When the occluder is to be inserted into the arterial system, a catheter is positioned by known methods into the desired area of the arterial system. The occluder 10D is compressed like an umbrella to a minimal diameter and pushed through the catheter by a wire or flexible rod 106. When the occluder 10D is pushed out of the end of the catheter, it expands outwardly like an umbrella opening to have the fabric pile 18C press against the inner luminal wall of the arterial system to form a seal. The stent 50 has a plurality of ribs 52 and sufficient fabric pile to form sufficient matrix to close off the arterial vessel from blood flow.

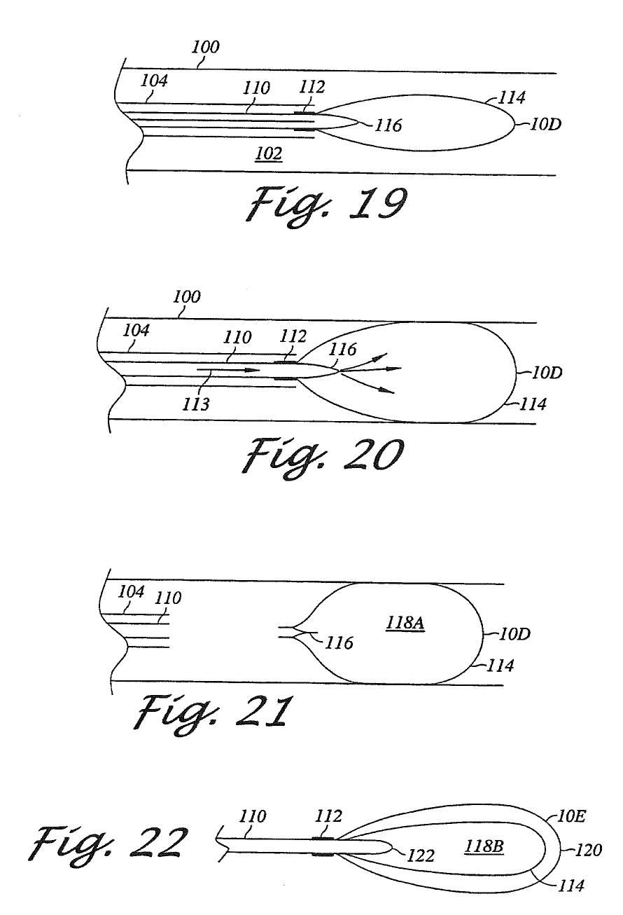

Referring to FIGS. 19-21, the procedure for utilizing one of the occluders of the present invention is illustrated showing its insertion, positioning and placement within an arterial vessel. A catheter 104 is first extended into the arterial system from a distal arterial branch by known methods. Once the catheter is positioned in a desired position, the occluder 10D which comprises semipermeable bag 114, having a one-way valve, such as a leaf valve 116 which is secured to a delivery tube 110 by a releasable bond 112, is inserted in the distal end of the catheter and pushed through the catheter out into the arterial stream. A slurry of particulate solid and saline or water is pumped through the delivery tube 110 through the leaf valve 116 into the interior of the bag 114. The particles can be made up of inert solid material such as silica particles and the like. A binder is included to bind the particles when in place. The binder is water activated. Preferably, at least some of the binder are particles of dehydrated hydrophilic gel. If inert particles are used, preferably a biocompatable adhesive is also placed in the slurry to bind the particles once they are in place in the bag 114. The use of dehydrated hydrophilic gel particles is preferred because once they are in the bag and have an opportunity to be hydrated by the water or saline, they expand, helping to fill or filling the bag and pushing the outer walls of the bag against the inner luminal wall of the arterial system. Once the surgeon makes the determination that the bag is filled through X-ray observation, delivery of the slurry is ceased and a slight suction is applied to the delivery tube to close off a one-way valve, such as a leaf valve 116. The releasable bond 112 is a material that will be dissolved or broken by the blood stream. Once the bond has dissolved or has been broken down by the blood stream, the delivery tube is withdrawn. Optionally, the delivery tube and the catheter can be removed simultaneously. Optionally, the bag can have an outer fabric pile similar to the graft and occluders shown in FIGS. 1-18. The bag can be a fabric bag or semipervious membrane which permits the diffusion of water across the bag envelope.

Referring to FIG. 22, an alternative embodiment occluder 10E is illustrated. Occluder 10E is utilized in the same manner as occluder 10D. However, some of the operational steps are different. Occluder 10E has an impervious outer bag 120, such as a rubber bag or other elastic flexible material bag. Within the bag, there is situated a semipermeable bag 114. Occluder 10E, when inserted into the catheter and inserted into the vascular system, has bag 114 filled with dehydrated hydrophilic gel granules 118B. When occluder 10E has been positioned in the arterial system in the same manner as occluder 10D, water is inserted through the delivery tube 110 through water pervious filter finger 122 into bag 114. The water hydrates the dehydrated hydrophilic gel 118B expanding the gel which expands bag 114 against bag 120 forcing the outer wall of bag 120 against the inner surface of the arterial vessel to occlude the vessel off. Water can migrate across the envelope of semipermeable bag 114 to permit bag 114 to completely fill the envelope bag 120. As in the same manner as occluder 10D, the blood stream dissolves or breaks down the detachable bond 112 permitting the delivery tube 110 to be freed from the occluder 10E.

The graft of FIGS. 1-3 and 13 and the occluders of FIGS. 4-12 and 14-16 are all delivered through catheters. The catheters are first positioned in the arterial system at the desired point of insertion of the graft or occluder. The distal end of the catheter is inserted into the arterial system through a distal branch of the arterial system. This will normally be one of the arteries in the legs or in the arms. Insertion and placement of the catheter is followed by known X-ray techniques. The graft of FIGS. 1-3 and 13 and the occluders of FIGS. 4-12, 15 and 16 are compressed when placed into the catheter. The compression compresses the stent and pushes down the fabric pile. The graft and occluders are pushed through the catheters employing wires having enlarged ends to engage the end of the graft or occluder without passing through the graft or occluder or with large rods which can engage the ends of the graft or occluder without passing through the graft or occluder. When the graft or occluder is pushed out of the proximal end of the catheter at the area where it is to be positioned and placed, the self-expanding stent expands circumferentially outward to expand the diameter of the graft and occluder to ensure contact between the fabric pile and the entire inner surface of the arterial vessel. Alternatively, expandable stents can also be employed. There may be situations because of the highly irregular shape of the interior wall of the arterial system that small gaps form. In those cases, the hydrophilic gel can assist in filling the gaps.

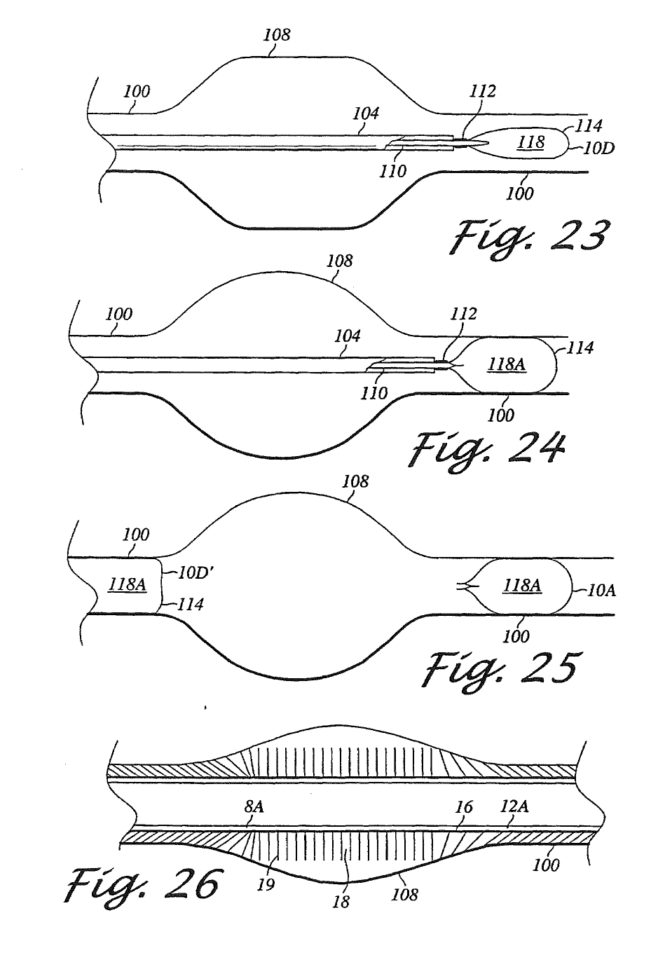

Referring to FIGS. 23-25, this illustrates a method for closing off an aneurysm which has been bypassed with a bypass vessel as is well known to the art. At the proximal end of the aneurysm in an area where the vascular wall has integrity and can be closed off is closed off, the vascular vessel is closed off with an occluder 10D although the occluders of 10A through 10E can also be used. The catheter 104 is positioned in the area where the occluder has to be positioned and placed, the occluder with its delivery tube 110 is inserted in the distal end of a catheter, pushed through the catheter and out the proximal end of the catheter at the desired location. The slurry of water and particulates are pumped through the delivery tube 110 into the bag 114 to enlarge the bag and have the bag complete occlude off the arterial vessel as described above. Preferably the slurry is a slurry of water or saline and hydrophilic gel particles. The surgeon waits for the blood to break apart or dissolve the detachable bond 112 to permit withdrawal of the delivery tube 112 and the catheter. The same operation is repeated to the vascular vessel at the distal end of the aneurysm. The surgeon chooses an area of the arterial vessel that has good integrity that can be safely and securely occluded. Occluder 10D′ is positioned and inplaced in the same manner as occluder 10D was positioned and inplaced at the proximal end of the aneurysm. Thus, by bypassing the aneurysm with known techniques and occluding off the aneurysm from the vascular system, the life-threatening problem associated with a bursting aneurysm is eliminated.

FIG. 26 shows the use of the stent graft 8A of the present invention used to bridge an aneurysm 108 in a vascular vessel 100. Stent graft comprises a tubular stent 12A which can be permanently expanded from a first diameter to a second larger diameter, an expandable graft 16 surrounding the tubular wall of the stent. The expandable graft has a fabric pile backing 16 of individual fibers 19. The stent graft is positioned in the vascular vessel, expanded to seat the stent graft in the vascular vessel and bridge the aneurysm 108. Individual fibers 19 of the fabric pile backing fill the gaps between the outer wall of the expandable graft and the inner wall of the vascular vessel. Preferably, the fabric pile has been coated with a hydrophilic polymeric gel which expands when wetted and fills the gaps.

I claim:

1. An endovascular stent for a vascular vessel comprising a generally tubular stent adapted to permanently expand from a first diameter to a larger second diameter, the stent made of at least one helically wound wire, each wire of the stent comprising at least two twisted strands, the twisted strands securing fibers to form a fiber pile extending outwardly from the stent.

2. The endovascular stent according to claim 1 wherein the twisted strands also securing fibers to form a fiber pile extending inwardly into the stent to substantially occlude the inner bore of the stent.

3. The endovascular stent of claim 1 wherein the fibers are coated with at least a partially dehydrated hydrophilic polymeric gel.

4. An endovascular stent for occluding the lumen of a vascular vessel comprising a generally conical-shaped stent member having a first large diameter at one end and a small diameter at the opposing end, the stent adapted to permanently expand the first large diameter to a larger diameter, the stent made of at least one helically wound wire, each wire of the stent comprising at least two twisted strands, the twisted strands securing fibers to form a fiber pile extending outwardly from the stent and extending inwardly into the stent to substantially occlude the inner bore of the stent.

5. The endovascular stent of claim 4 wherein the fibers are coated with at least a partially dehydrated hydrophilic polymeric gel.

6. An endovascular stent for occluding a lumen of a vascular vessel comprising a generally umbrella-shaped member having a plurality of radially wire ribs joined together at one of their ends and biased to extend radially outwardly and downwardly, each rib comprising of at least two twisted strands, the twisted strands securing fibers which extend outwardly from the wire ribs to form a fiber pile.

7. The endovascular stent according to claim 6 wherein the twisted strands also secure fibers which extend inwardly from the wire ribs to substantially occlude the inner bore of the stent.

8. The endovascular stent according to claim 6 wherein the fibers are coated with at least a partially dehydrated hydrophilic polymeric gel.

9. An endovascular stent comprising a generally tubular-shaped stent adapted to permanently expanded from a first diameter to a larger second diameter, the stent surrounded on its external tubular walls with a radially expandable graft having a fiber pile extending outwardly from the walls of the graft.

10. The endovascular stent according to claim 9 wherein one end of the stent is covered by an end wall of the graft, the end wall of the graft having a fiber pile extending outward from its external surface.

11. The endovascular stent according to claim 9 wherein the radially expandable graft having end walls at both ends and sealing off the ends of the stent, the end walls of the graft having fiber pile extending outwardly from their external surface.

12. The endovascular stent according to claim 9 wherein the fiber pile is coated with at least a partially dehydrated hydrophilic polymer gel.

13. An occluder comprise a semi-permeable expandable bag having a one-way flow valve which is secured to a delivery tube by releasable bond, the bag having a chamber which is in fluid communication with the delivery tube via the one-way flow valve, a slurry of inert biocompatible particulate solids at least partially filling the bag, particulate solids comprising at least a partially dehydrated hydrophilic polymeric gel that when wetted with water expands filling the bag's chamber and expanding the bag radially outwardly, the chamber adapted to receive aqueous solutions through the delivery tube and one-way flow valve.

14. An occluder comprising a semi-permeable bag having a central chamber, a one-way flow valve opening into the central chamber, a delivery tube which is secured to the one-way flow valve for delivery of materials through the valve into the central chamber, the delivery tube secured to the bag by releasable bond, the bag adapted to receive a slurry of particulate solids in an aqueous medium through the delivery tube and through the one-way valve.

15. An occluder comprising an impervious expandable outer bag and an expandable semi-permeable inner bag within the impervious outer bag to form a chamber between the inner side of the outer bag and the outer side of the inner bag, a one-way flow valve within the inner bag adapted to permit delivery of aqueous solutions into the inner bag through a delivery tube secured to the one-way flow valve and in fluid communication with the valve, the delivery tube is secured to the outer bag by a releasable bond, particulate solids filling the inner bag, the particulate solids comprising at least a partially dehydrated hydrophilic polymeric gel which is adapted to expand when hydrated.
